(19) United States
(12) Patent Application Publication
(10) Pub. No.: US 2020/0246633 A1
(43) Pub. Date: Aug. 6, 2020

(54) DEUTERON THERAPY

(71) Applicant: Robert G. Zamenhof, Brookline, MA (US)

(72) Inventor: Robert G. Zamenhof, Brookline, MA (US)

(21) Appl. No.: 16/782,408

(22) Filed: Feb. 5, 2020

Related U.S. Application Data
(60) Provisional application No. 62/801,787, filed on Feb. 6, 2019.

(51) Int. Cl. A61N 5/10; A61F 9/007

(52) U.S. Cl. CPC A61N 5/10; A61N 5/1077; A61N 2005/1095; A61N 2005/1087; A61F 9/0079

(57) ABSTRACT

Disclosed is a method of utilizing deuterons (nuclear particles consisting of a proton and a neutron) for charged particle radiotherapy. Compared with proton therapy, at their maximum treatment depth of 66 mm, 125 MeV deuterons possess 82-85% less beam straggling than protons. This difference enables better protection of radiosensitive critical tissues that may be in contact with a tumor. Alternatively, it enables higher doses to be delivered to the tumor, resulting in better tumor control. The implementation of deuteron therapy interchangeably alongside proton therapy requires minor modifications at modest cost to many existing proton therapy systems and provides a clinically useful hybrid particle therapy facility. A free-standing deuteron therapy facility that employs only deuterons is also described.

### RELATED APPLICATIONS

This application claims the benefit of priority to U.S. Provisional Patent Application No. 62/801,787, filed on Feb. 6, 2019.

### FIELD OF THE INVENTION

The field of the invention is radiotherapy for cancer, delivered by a charged particle beam.

### BACKGROUND OF THE INVENTION

Over the past 20 years, proton therapy has superseded mega-voltage x-ray therapy as a more precise form of radiotherapy due to better dose conformity and the absence of exit dose. ‘Dose conformity’ refers to the ability of a radiation to deliver dose to a tumor volume with a minimum amount of dose ‘spillover’ to adjacent normal tissues. The absence of ‘exit dose’, an important feature of all charged particle beams, protects normal organs and tissues downstream from the targeted tumor. High dose conformity is important in proton treatments where dose spillover could pose a serious risk to the patient, such as in the treatment of ocular tumors, pediatric spinal cord and brainstem tumors, and other relatively superficial tumors adjacent to critical normal tissues. The recent introduction of carbon-ion therapy has greatly improved dose conformity relative to protons, and provides superior radiobiological characteristics for further improved tumor control. Unfortunately, facilities for carbon-ion therapy, although presently considered in the industry to be the ‘gold standard’ for dose conformity, are exceedingly expensive to construct, operate, and maintain.

Numerous clinical studies comparing protons and carbon ions have demonstrated significant clinical advantages of carbon ions¹. This advantage is primarily due to two factors: 1) the superior dose conformity of carbon ion beams, and 2) the higher linear energy transfer (LET) and lower oxygen enhancement ratio (OER) of carbon-ions near the end of their range. The high LET produces proportionally more DNA double-strand breaks, which are generally not repairable by rapidly dividing tumor cells, while low OER provides improved control of tumors that have hypoxic components. Of all the experimental particle therapies mentioned, protons and carbon-ions are probably the most clinically successful, although fast neutron therapy has shown itself to be useful in limited clinical situations, but is also extremely costly to construct and operate.

Other forms of heavy particle therapy have been tried experimentally over the past few decades; including alpha particles, pi mesons, and boron neutron capture. However, none of these experimental treatment modalities has earned approbation by the medical community or adequate financial support from the U.S. government for development as mainstream tools of radiation therapy.

### SUMMARY OF THE INVENTION

The present disclosure provides a device, comprising an ion source and an energy selector device; wherein said ion source produces protons and deuterons.

In some embodiments, the ion source is a Penning ionization gauge (PIG).

In some embodiments, the ion source emits deuterons.

In some embodiments, the energy selector device is a pre-absorber.

In some embodiments, the energy selector device comprises two wedges of material configured to move with respect to each other in a way that allows a selectable variable path distance for either a proton or a deuteron beam passing through the pre-absorber.

In some embodiments, the energy selector device comprises graphite.

In some embodiments, the energy selector device comprises beryllium.

In some embodiments, the present disclosure relates to any one of the aforementioned devices, further comprising a scattering foil.

In some embodiments, the scattering foil comprises lead.

In some embodiments, the scattering foil comprises nickel.

In some embodiments, the present disclosure relates to any one of the aforementioned devices, further comprising a range modulator wheel.

In some embodiments, the present disclosure relates to any one of the aforementioned devices, further comprising a compensator.

In some embodiments, the present disclosure relates to any one of the aforementioned devices, further comprising a cyclotron.

In some embodiments, the cyclotron is a momentum-bound cyclotron.

In some embodiments, the cyclotron is a synchrocyclotron.

In some embodiments, the PIG is positioned inside the cyclotron.

In some embodiments, the energy of the deuteron beam is from 60 MeV to about 250 MeV. In some embodiments, the energy of the deuteron beam is from about 100 MeV to about 200 MeV. For example, the energy of the deuteron beam is about 125 MeV.

In certain embodiments, the present disclosure relates to a method of treating a disorder, comprising administering to a tissue of a subject in need thereof an effective amount of a deuteron beam generated with the aid of any one of the aforementioned devices.

In some embodiments, the disorder is cancer.

In some embodiments, the tissue is cancerous.

In some embodiments, the cancer is selected from the group consisting of an ocular tumor, orbital tumor, lacrimal gland tumor, salivary gland tumor, intracranial falx meningioma, intracranial occipital meningioma, acoustic neuroma, shallow soft-tissue sarcoma, bone sarcoma of the extremities, neck lympho-nodal disease, chest wall desmoid tumor, abdominal wall desmoid tumor, breast cancer, pediatric rhabdomyosarcoma, and head and neck cancer.

In some embodiments, the disorder is head and neck cancer; and the head and neck cancer is selected from the group consisting of larynx cancer, thyroid cancer, salivary gland cancer, uveal melanoma, and retinal metastases.

In some embodiments, the deuteron beam is administered to an eye of the subject.

In some embodiments, the disorder is age-related macular degeneration, or a benign retinal tumor.

### BRIEF DESCRIPTION OF THE DRAWINGS

FIG. 1 depicts the concept of ‘pre-absorption’ of a particle's range as the equivalent in a momentum-bound cyclotron of changing the particle's penetration depth in tissue. Desired treatment depth is S. Cyclotron's fixed energy produces a particle penetration depth of D. The pre-absorber of thickness P ‘pulls back’ the particle's range to depth S. To do this, S=D−P. And, since D=P+S, straggling at penetration depths D and S is the same.

FIG. 2 is a (a) tabular summary and (b) graphic summary of particle beam energy, corresponding range in water, and end-of-range lateral and longitudinal straggling parameters for protons, deuterons, and carbon ions.

FIG. 3A shows proton and carbon ion comparative treatment plans and dose-volume histograms for a skull-based chordoma tumor, demonstrating the advantage of a particle beam with low straggling parameters. Top images are carbon ion, bottom images are proton. The four small circles in the top-left panel are the sampling volumes defined for determining dose to the brainstem to which spill-over dose must be avoided.

FIG. 3B shows the dose-volume histograms that quantify the difference in brainstem dose delivered by protons vs. carbon-ions (roughly equivalent in this example to deuterons). Carbon ion spillover dose to the brainstem is 3.1 times less than for protons, normalized to equal dose to tumor.

FIG. 4 is a representation of the subset of conditions that could be advantageously treated using deuterons rather than protons. Subset characteristics are shallow tumors that have adjacent critical normal tissues.

FIG. 5 depicts proton beam-line devices in a passive scattering system that need to be modified for deuteron operation; including scattering foil, energy selector, modulator wheel, compensator, and treatment planning system.

FIG. 6 is a diagram of a Penning-type (PIG) ion source that can be filled with light hydrogen or with heavy hydrogen (deuterium), depending on whether proton or deuteron operation is desired. When activated, anodes and cathodes embedded in the gas volume of the PIG initiate a flow of low-energy electrons which ionize the gas producing positively-charged deuterons or protons that are then extracted through the extraction slit and accelerated by the cyclotron's magnetic field.

FIG. 7 depicts a conceptual design of an energy selector for providing depth-of-penetration control for protons and deuterons using a single device.

FIG. 8 is a depth-dose curve for a mono-energetic or ‘pristine’ 250 MeV proton beam showing plateau and Bragg peak features.

FIG. 9 depicts production of a spread-out-Bragg-peak (SOBP) using a range-modulator wheel. The series of curves at the bottom of the figure are six (6) pristine proton Bragg peaks with weighted intensities shifted in depth by the step thickness changes in the range modulator wheel. The SOBP (spread-out-Bragg-peak), or ‘cumulative total’, is the summation of the six (6) individual pristine Bragg peaks.

FIG. 10 is an illustration of (a) a range modulator wheel and (b) photograph of a physical range modulator wheel. The small circle in (a) represents the instantaneous footprint of the partially scattered proton beam as the range modulator wheel rotates. Step thicknesses determine the depth of the resulting SOBP, while step widths determine the relative weights—depicted in FIG. 9 as heights—of the array of pristine Bragg peaks to produce a flat SOBP.

FIG. 11 is a graph of relative biological effectiveness (RBE) vs. linear-energy-transfer (LET) for protons and deuterons (and alpha particles) from irradiation of V-79 cells. In both the Bragg peak (domain 1) and in the plateau (domain 2) regions the RBEs for protons and deuterons are essentially identical.

FIG. 12 is a graph of ‘breakup’ and ‘Pickup’ cross-sections for deuterons vs. deuteron energy in MeV/nucleon. For a 125 MeV deuteron, breakup cross-section is ˜14 mB, pickup cross-section is ˜34 mB, and total ‘deuteron-disappearance’ cross-section is ˜48 mB. Deuteron breakup produces a proton and a neutron while deuteron pickup produces a triton (a nucleus of tritium).

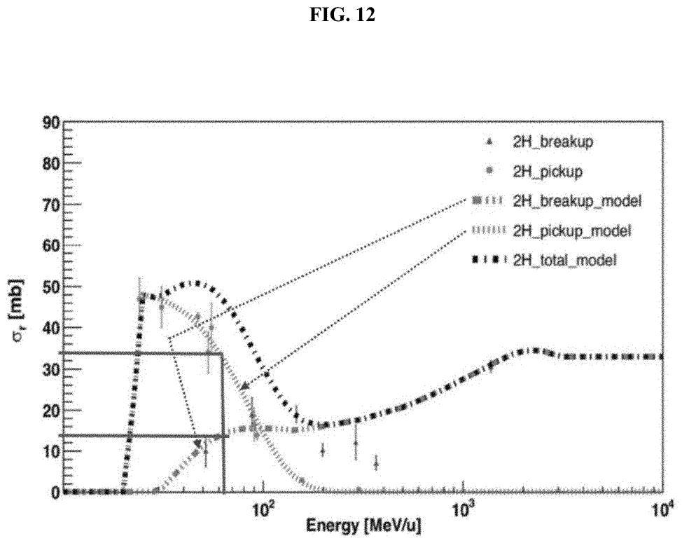

### DETAILED DESCRIPTION

The detailed description is provided to assist the reader in gaining a comprehensive understanding of the devices and methods described herein. Accordingly, various changes, modification, and equivalents of the devices and methods described herein will be suggested to those of ordinary skill in the art. The progression of fabrication operations described are merely examples, however, and the sequence type of operations is not limited to that set forth herein and may be changed as is known in the art, with the exception of operations necessarily occurring in a certain order. Also, description of well-known functions and constructions may be omitted for increased clarity and conciseness.

Note that spatially relative terms, such as “up,” “down,” “right,” “left,” “beneath,” “below,” “lower,” “above,” “upper” and the like, may be used herein for ease of description to describe one element or feature's relationship to another element(s) or feature(s) as illustrated in the figures. Spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

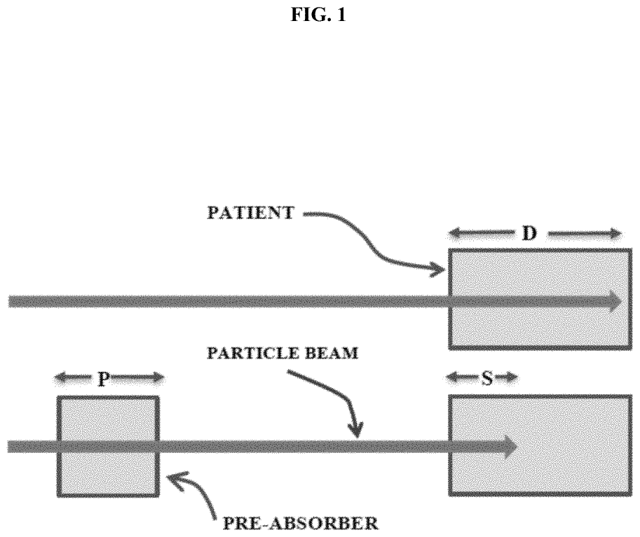

Proton radiotherapy has established itself as a valuable tool in the radiation treatment of cancer, primarily due to its superior dose conformity compared to megavoltage x-rays, mostly by virtue of the lack of greatly reduced exit dose compared to megavoltage x-rays. This has made protons the modality of choice over megavoltage x-rays for many therapeutic applications, including prostate cancer (where the incidence of post-treatment rectal bleeding is significantly diminished) and childhood brain tumors (where the incidence of mental retardation is also significantly diminished). Proton therapy systems are currently designed to treat patients to depths of up to approximately 30-38 cm. However, because ‘momentum-bound’ medical cyclotrons operate with fixed magnetic fields and, therefore, with fixed proton energies, mechanical devices such as ‘energy selectors’ (also referred to as ‘energy pre-absorbers’) must be utilized to pull back the proton range to provide variable depths of penetration. A 250 MeV proton beam, for example, has a fixed depth of penetration of approximately 374 mm in water (very similar to soft tissue). If shallower depths need to be treated, a corresponding ‘pre-absorber’ must be deployed to reduce the energy of the protons before they enter the patient. However, since in a momentum-bound cyclotron the particle acceleration energy is fixed, the resulting total proton penetration depth (i.e., pre-absorber+patient) must remain at 374 mm. This concept is illustrated in FIG. 1. As protons penetrate a medium they experience millions of small random trajectory deviations mainly due to coulombic scattering with the electrons of the medium. These deviations statistically combine so that upon reaching their maximum penetration depth the protons are spread out in their lateral and longitudinal dose envelopes. ‘Range-straggling’, as this phenomenon is called, is normally quantified using a standard deviation metric.

Subatomic particles called deuterons can be used to deliver therapy to cancer patients with ocular and other relatively superficial tumors where the amount of tumor dose that can be delivered may be restricted by the risk of over-irradiating adjacent critical normal tissues. Many malignant conditions fall into this category, including uveal melanomas, retinal metastases, orbital tumors, pediatric rhabdomyosarcomas, lacrimal gland tumors, larynx, thyroid, and salivary gland tumors, and many others.

Deuteron therapy would provide substantially higher dose conformity to tumor volumes than is currently attainable with proton beams, and in fact closely approaches the high ‘gold standard’ of dose conformity exhibited by carbon-ions. However, unlike carbon-ions, deuterons can be implemented within existing technology utilized by many current proton therapy systems, or as ‘stand-alone’ particle therapy systems specifically designed to treat disease conditions that demand exceptionally precise dose conformity, at a cost that is still at least ten times lower than the cost to build a de novo carbon-ion facility. Based on 2017 statistics, there were 75 active proton treatment facilities worldwide with 41 under construction. In comparison, due to the enormously higher technological complexity and capital cost, there were only 10 carbon ion treatment facilities worldwide with 4 under construction.

Relatively minor modifications to a ‘momentum-bound’ cyclotron (the type frequently used to deliver proton therapy) and to its associated equipment and software are necessary to enable the use of deuterons interchangeably with protons utilizing a single cyclotron. Alternatively, a dedicated deuteron treatment facility can be constructed using a separate cyclotron. A deuteron beam enables the treatment of tumors to a depth of 66 mm, with lateral and longitudinal straggling (beam ‘fuzziness’) reduced by 82-85% compared to a proton beam at the same treatment depth.

From a radiobiological perspective, the implementation of deuterons should not require any modification in clinical dose prescription, since their radiobiological properties are essentially identical to those of protons. The ability of deuterons to deliver tumor dose with much higher conformity than is possible with protons also permits prescribed doses to tumor to be increased while maintaining dose to adjacent normal tissues at a safe level, a condition that leads to improved therapy outcomes.

Deuterons are sub-atomic particles, consisting of a proton bound to a neutron. Because its mass is twice that of a proton, for the same initial energy a deuteron penetrates a material (e.g., tissue) to a shallower depth than a proton. For example, a 250 MeV proton has a penetration depth in water of 374 mm, while a 250 MeV deuteron has a penetration depth in water of only 226 mm. When accelerated by a momentum-bound cyclotron designed to accelerate 250 MeV protons, the range of a 125 MeV deuteron in water is 66 mm (calculated using the SRIM-2008 charged particle transport code (1)). Although 66 mm would appear to be somewhat shallow, there are a large number of clinical applications where a 66 mm penetration depth or less would be adequate. The big advantage of deuterons over protons, however, is that because of their inherently shorter range they require much less pre-absorption. Consequently, a deuteron's very low straggling parameters reflect required pre-absorption thicknesses of just 0-66 mm rather than 0-374 mm, as would be required if a pre-absorbed proton beam were used to treat to a depth of 0-66 mm.

Therefore, the advantage of deuterons over protons is that for relatively shallow tumors that may be juxtaposed with critically radiosensitive normal tissues, their very small straggling parameters compared to protons would result in safer treatments because of less dose ‘spillover’ to normal tissues. A very important concept is that due to the lower amount of dose spillover, the use of deuterons would permit a corresponding increase in tumor dose while maintaining critical normal tissue dose at the same levels as when using protons, a maneuver that would result in superior tumor control.

250 MeV protons have the same momentum as 125 MeV deuterons; therefore, many 250 MeV momentum-bound cyclotrons could also accelerate 125 MeV deuterons. This is evidenced in the brochures of the IBA Cyclotron Corporation, a major supplier of cyclotrons for proton therapy and for radiopharmaceutical production. For example, IBA's 17 MeV cyclotron can also accelerate 8.3 MeV deuterons, their 30 MeV proton cyclotron can also accelerate 15 MeV deuterons, and their variable magnetic-field cyclotron can accelerate 10-40 MeV protons or 5-20 MeV deuterons. These cyclotrons, however, are designed specifically for radiopharmaceutical production and do not produce adequate energy to be used for particle therapy, where relativistic effects might introduce some complications regarding the combined acceleration of protons and deuterons by a single cyclotron.

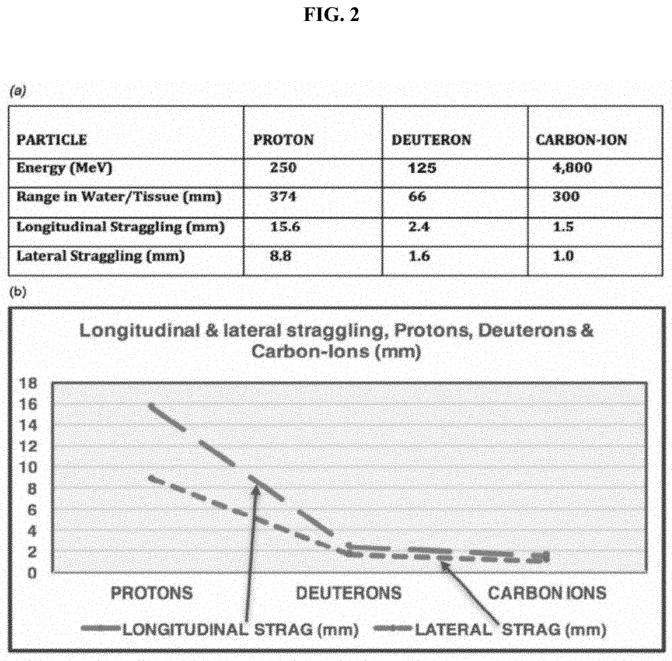

FIG. 2 (a) compares the straggling parameters of protons, deuterons, and carbon-ions. The straggling parameters were calculated using the SRIM-2008 charged particle transport code (1). The very large improvement in straggling exhibited by deuterons compared to protons is shown in the last two rows of FIG. 2 (a), where straggling parameters for deuterons are approximately 82-85% lower than for protons, and in fact approach those of carbon-ions, which are currently considered to be the ‘gold standard’ for dose conformity in particle therapy.

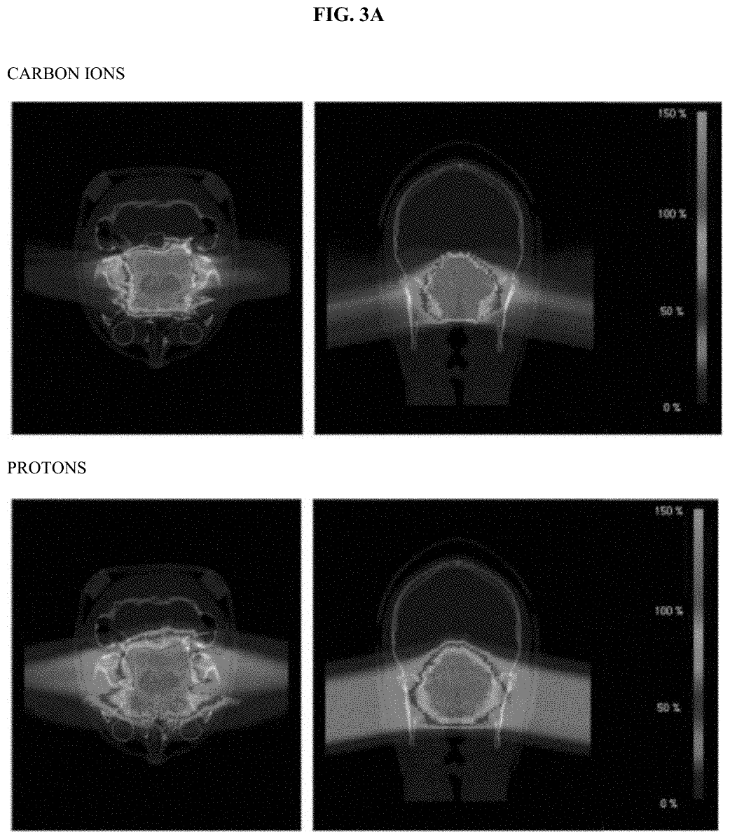

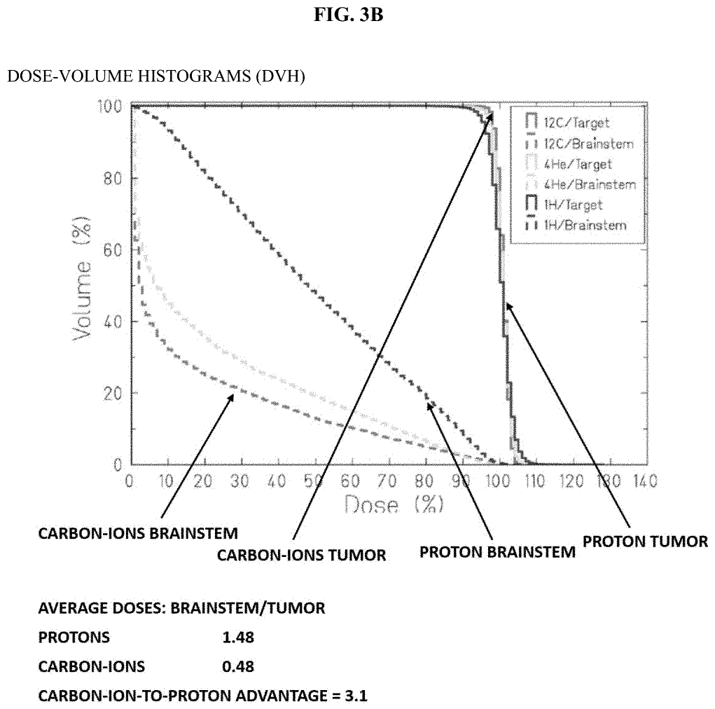

The clinical importance of treating tumors that are adjacent to critical tissues with a minimum amount of dose spillover is illustrated by the following treatment plan comparison2. The tumor being treated is a skull-based chordoma, with the brainstem representing adjacent critical tissue. FIG. 3A compares treatment plans for protons and carbon-ions. Because deuterons are not, nor have ever been used for radiation therapy, no explicit deuteron treatment plans exist that could be included in this comparison. However, based on the straggling data shown in FIG. 2, deuterons possess very similar straggling parameters to carbon-ions, so the comparative treatment plans shown in FIG. 3A could legitimately be considered as approximating a treatment plan comparing protons and deuterons from the perspective of relative dose conformity.

The top two panels of FIG. 3A, labeled ‘carbon-ions’, show a treatment plan for the skull-based chordoma using parallel-opposed carbon ion beams. The four small circles represent the sampling volumes that are used to determine the dose to the brainstem. The bottom two panels, labeled ‘protons’, show an equivalent treatment plan using parallel-opposed proton beams. In both cases the left panels show axial planes and the right panels show sagittal planes through the anatomical region. Comparing the two axial panels on the left, it is evident, even visually, that the brainstem receives a substantially higher dose in the proton treatment plan than it does in the carbon ion treatment plan. To quantify this difference, ‘dose-volume histograms’ (DVH) for the two treatment plans are shown in FIG. 3B. The coverage of the tumor is equally effective for both proton and carbon ion treatment plans, as demonstrated by the superimposed DVH curves labeled ‘proton tumor’ and ‘carbon-ion tumor’. However, the two DVH curves representing the brainstem dose are very different, shown by the DVH curves labeled ‘protons brainstem’ and ‘carbon-ions brainstem’. In fact, the average brainstem dose in the proton treatment plan is about 3 times higher than in the carbon ion treatment plan. Consequently, assuming the dose conformity for deuterons to be roughly equivalent to the dose conformity for carbon ions, deuteron treatment of the skull-based chordoma would substantially reduce dose to the brain-stem compared to proton treatment. Alternatively, this would enable significantly higher tumor doses to be delivered to the tumor resulting in substantially better tumor control.

### Implementation of Deuterons Interchangeably Alongside Protons to Enable Use of the Most Advantageous Particle for Every Clinical Situation.

A major additional advantage of deuterons over protons, beyond their advantage of dose conformity, is that many proton facilities currently employ momentum-bound cyclotrons, and could be modified to also accelerate deuterons at relatively moderate cost. Therefore, it is proposed that 1) deuteron therapy could be provided interchangeably alongside standard proton therapy using the same cyclotron and associated beam transport equipment with relatively minor modifications, thereby permitting the greatest clinical flexibility in treating a wide variety of tumors using protons or deuterons; or 2) deuteron therapy could be provided for specific therapeutic applications using a dedicated cyclotron. Under either of these scenarios, protons would generally be used for deep treatments and/or larger tumor volumes, while deuterons would be used for shallow treatments where dose spillover to critically sensitive adjacent normal tissues could pose a significant risk to the patient.

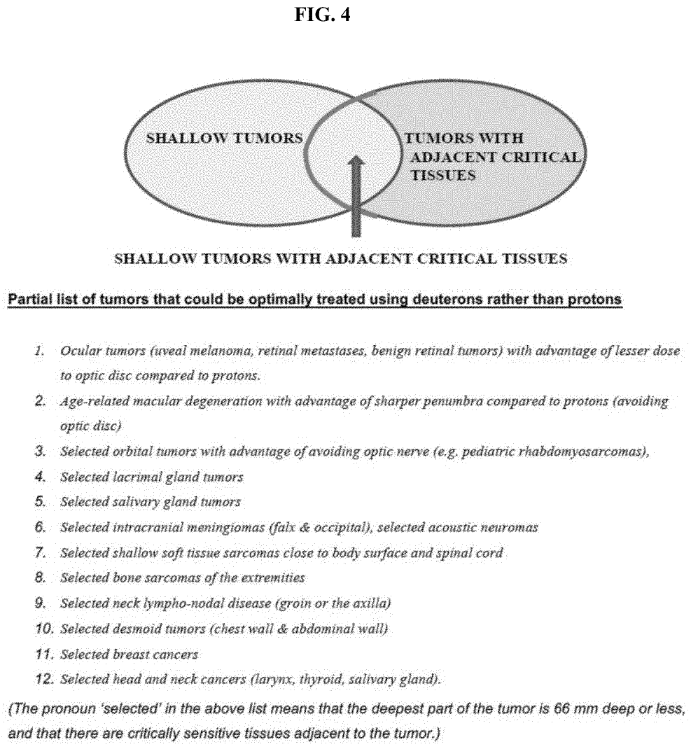

FIG. 4 illustrates the subset of disease conditions that could be advantageously treated using deuterons. The intersection between the two ovals labeled ‘shallow tumors’ and ‘tumors with adjacent critical tissues’ represents the subset ‘shallow tumors with adjacent critical tissues’. It is this subset of clinical conditions where the choice of deuterons would be most advantageous. FIG. 4 also provides an expanded list of treatment sites that could potentially be treated more effectively or more safely with deuterons.

### Equipment Modifications Necessary for Deuteron Operation.

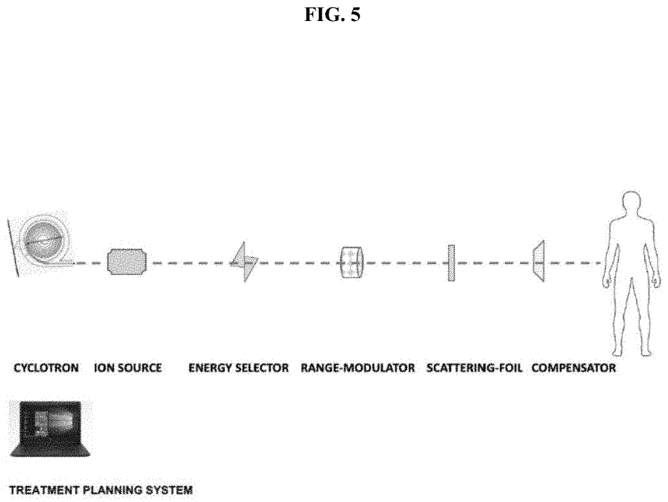

To better understand the proton therapy equipment modifications that would be needed to provide deuteron therapy, it would be useful to review the primary components of a proton therapy beam-line. FIG. 5 shows the basic devices present in a proton therapy beam-line in a ‘passive scattering’ proton therapy system. Some more recent proton therapy technologies provide ‘spot-scanning’, which from the perspective of enabling deuteron operation would involve even fewer modifications than passive scattering. However, not all particle accelerators used for proton therapy are momentum-bound cyclotrons that could accelerate both protons and deuterons. In those cases, a dedicated deuteron therapy facility employing a separate cyclotron could be implemented. Synchrotrons or synchro-cyclotrons are capable, to varying extents, of modulating the energy of the accelerated particles and could, therefore, dispense with at least the energy-selector, range-modulator, and compensator components of the beam line. For such systems, the straggling properties of deuterons may not be significantly better than those of protons, since the protons could be adjusted to shallower depths not by using physical pre-absorbers but by using reduced acceleration energies. Nevertheless, largely for reasons of capital cost, whether an installed proton therapy system could accelerate both protons and deuterons or whether deuterons would have to be accelerated by a separate cyclotron, the construction and operation costs of either of these approaches would still be far lower than the construction and operation cost of a carbon-ion facility.

The following US patents and patent applications, each of which is incorporated by reference for the cited disclosure, disclose devices and methods related to ion beam radiation therapy:

U.S. Pat. No. 4,139,777 discloses a cyclotron and a therapy installation;

U.S. Pat. No. 4,870,287 discloses a proton beam therapy system for selectively generating and transporting proton beams from a single proton source and accelerator to a plurality of patient treatment stations;

U.S. Pat. No. 6,814,694 discloses an apparatus for carrying out proton therapy comprising a proton beam guiding device using magnets and quadrupoles, and a control device with an exit window for guiding or directing the proton beam to the treatment spot in the patient;

U.S. Pat. No. 10,039,935 discloses a processor configured to control relative movement between the proton beam and the patient in two dimensions, and to control the proton energy distribution to adjust the penetration depth of the protons in the third dimension;

U.S. Pat. No. 6,683,318 discloses ion beam therapy system that allows well focused pencil-like beams of charged particles with an adjustable spot-size to be formed and scanned over the treatment field following the tumor contours;

U.S. Pat. No. 6,800,866 discloses an accelerator system having a wide ion beam current control range and capable of preventing an excessive dose of irradiation from being erroneously transported to the downstream side;

U.S. Pat. No. 6,809,325 discloses an apparatus for generating, extracting and selecting ions used in a heavy ion cancer therapy facility;

U.S. Pat. No. 7,560,715 discloses a method for implementing intensity-modulated proton therapy on a predetermined tumor volume within the body of the patient;

U.S. Pat. No. 9,757,592 discloses systems and apparatuses for providing particle beams for radiation therapy with a compact design;

U.S. Pat. No. 9,962,560 discloses a particle accelerator to output a particle beam; and a scanning system for the particle accelerator to scan the particle beam across at least part of an irradiation target;

US 2011/0127443 discloses integrated assembly incorporating both aperture material and range compensator material for profiling, shaping, and modulating a proton beam; and

U.S. Pat. No. 7,208,748 discloses a charged particle beam scatterer/range modulator comprising a fluid reservoir having opposing walls in a particle beam path and a drive to adjust the distance between the walls of the fluid reservoir under control by a programmable controller.

Each of the U.S. patents and patent applications listed above is incorporated by reference. Aspects of the aforementioned devices and methods are suitable or can be adapted for the present invention.

1. Modification of the cyclotron. Most cyclotrons currently employed for proton therapy are momentum-bound, so as long as the accelerated particles conform to the fixed momentum requirement, the cyclotron is able to accelerate particles of varying masses—for example, deuterons at half the energy of protons. This also applies to magnetic and electrostatic deflection systems, including complex beam-steering functions such as focusing and beam scanning. Therefore, necessary modifications to momentum-bound cyclotrons to enable deuteron operation could be accomplished at relatively modest cost.
A cyclotron comprises:
(a) a pair of opposed, spaced pole-shoes having their adjacent inner surfaces defining an accelerator zone,
(b) an electromagnetic coil system,
(c) at least one hollow accelerating ‘dee’ electrode positioned in the accelerator zone, and having a radio frequency resonator associated therewith,
(d) a magnet yoke shaped to substantially enclose the accelerator and constitute an ion attenuation shield for ions accelerated in the cyclotron,
(e) a vacuum chamber enclosing the accelerator zone and each ‘dee’ electrode,
(f) means for providing charged particles for acceleration within the accelerator zone,
(g) a target zone for a target device, and
(h) an ion beam outlet in the magnet yoke for emission of an ion beam produced in the cyclotron.

A cyclotron can be a synchrocyclotron with a magnet structure that has a field strength of at least about 6 Tesla, and the field strength can be from about 6 to 20 Tesla. The magnet structure can include superconducting windings that are cooled by cryocoolers.

A synchrotron can be an accelerator including a deflecting electromagnet arranged on a circulating orbit of a charged particle beam, four-pole divergence electromagnets and four-pole convergence electromagnets arranged on said circulating orbit. A high frequency waveform generating unit arranged on the circulating orbit for applying a high frequency electromagnetic field to the circulating charged particle beam circulating and for increasing the amplitude of oscillation of the particle beam to a level above a stability limit of resonance. A first deflector for beam ejection arranged on the circulating orbit for deflecting the charged particle beam excited above the stability limit of the resonance by the high frequency waveform generating unit and a second deflector for beam ejection arranged on the circulating orbit used in pairs with the first deflector for beam ejection for introducing the charged particle beam deflected by the first deflector for beam ejection into an ejected beam transporting system. The deflecting electromagnet and the second deflector for beam ejection are arranged in this order downstream with respect to the first deflector for beam ejection, and the four-pole divergence electromagnets are arranged downstream with respect to the first deflector for beam ejection and upstream with respect to the deflecting electromagnet and downstream with respect to the deflecting electromagnet and upstream with respect to the second deflector for beam ejection.

An example of a system for adjusting the energy level of a proton beam provided by a cyclotron is disclosed in U.S. Pat. No. 9,789,341 (incorporated by reference).

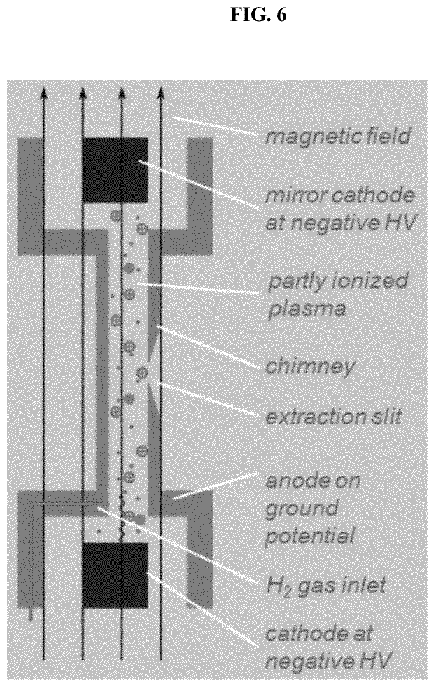

2. Modification of the ion source. FIG. 6 shows a diagram of a ‘Penning ionization gauge’ (or PIG) ion source, located at the hydrogen ion injection point near the center of a cyclotron. The diagram shows a single PIG under two conditions: 1) containing deuterium gas, for deuteron operation, and 2) containing light-hydrogen gas, for proton operation. When activated, anodes and cathodes embedded in the gas volume of the ion source initiate the flow of low-energy electrons, which ionize the gas contained in the PIG producing either positively-charged deuteron or positively-charged hydrogen ions that can be accelerated by the cyclotron's magnetic field. A single PIG ion source would need to be suitably flushed with deuterium gas or light hydrogen gas respectively prior to switchover to proton or deuteron operation.

The diagram for a PIG is shown in FIG. 6. The Penning gauge is a cold cathode type ionization gauge consisting of two electrodes, anode, and cathode. The outer cylinder of the gauge is the cathode and is at room temperature. The anode consists of a tungsten wire mounted in the center of the tube. A potential difference of about 2 to 3 kV is applied between anode and cathode through current limiting resistors. A magnetic field is introduced at right angles to the plane of the electrodes by a permanent magnet having nearly 80 milli-Tesla magnetic field which increases the ionization current.

The electrons emitted from the cathode (gauge head body) of the gauge head are deflected by means of a magnetic field applied at right angles to the plane of the electrodes and are made to take a helical path before reaching the anode loop. Thus, as they follow the helical path, the electrons ionize the gas by collisions, even at low gas pressures.

3. Modification of the scattering foil. Accelerated protons or deuterons leaving the exit port of a cyclotron do so as a very narrow (sub-millimeter) ‘pencil-beam’ of particles. In passively-scattered proton systems, this pencil-beam of particles must eventually be spread out to accommodate the field sizes required clinically (typically up to 30 cm×30 cm). The optimum design of multi-material scattering foils to optimally scatter deuterons and protons may differ, and thus may require actuators or a carousel arrangement to interchange them for proton or deuteron operation.

A cyclotron may comprise first and second scatterers. The first scatterer comprises a pair of parallel and opposing tapered wedges. The wedges are made of a high-Z material (e.g. lead, gold, nickel, etc.), which contains high atomic number atoms that generate scattering of the particle beam but only minimally change its energy. Each wedge slides back and forth on rails or shafts and is controlled by a motor and position control unit, so that equivalent sections of the wedges overlap at the beam's footprint position.

The second scatterer is mounted on a carousel and intercepts the path of the particle beam at a location downstream from the first scatterer. The second scattering foil is made of both high-Z material and low-Z material. The low-Z material (e.g. plastics, carbon, etc.) contains low atomic number atoms that do not generate substantial scattering but are intended to reduce the particle beam's energy. The high atomic number material has a cross-sectional profile that is approximately Gaussian in shape. The low atomic number material surrounds the high atomic number material and, directly beneath it, has a hollowed-out portion which has a mating profile that is approximately Gaussian in shape.

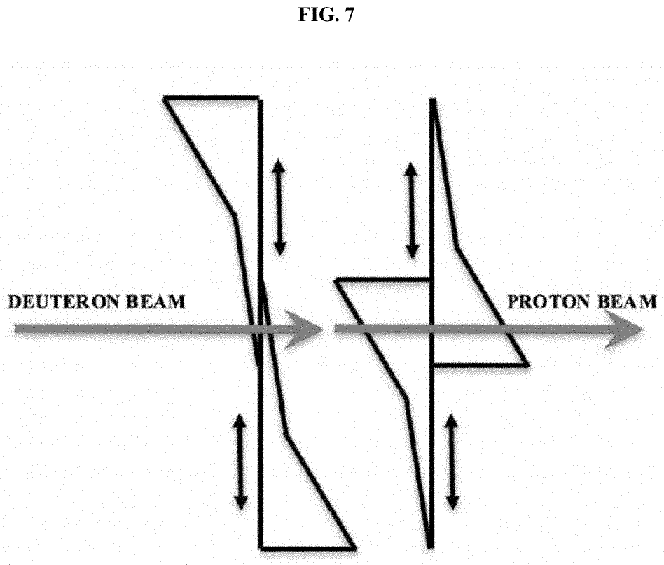

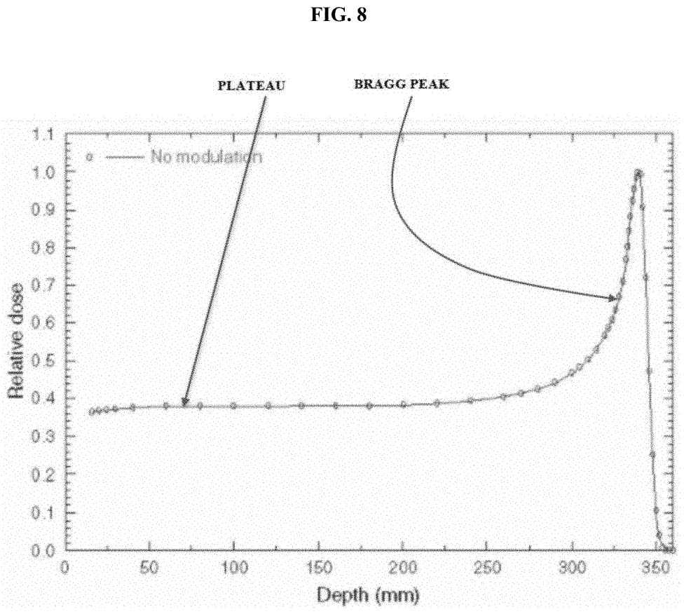

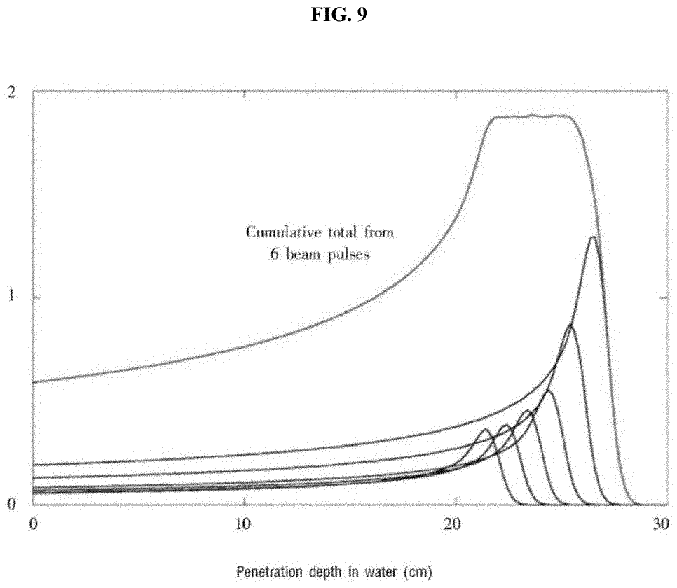

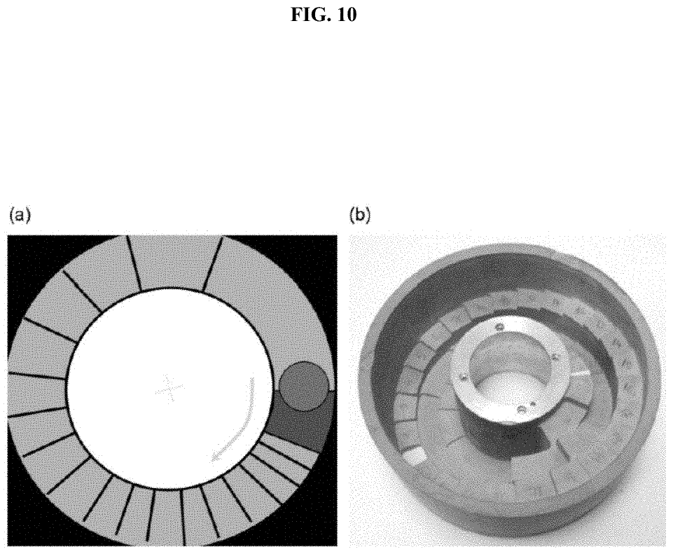

4. Modification of the energy selectors. To ensure that protons entering tissue possess the correct energy to reach a specific treatment depth, the fixed proton energy in a momentum-bound cyclotron must be adjusted to this required energy. In the case of passively scattered proton beams, this is usually accomplished using an ‘energy selector’ or ‘pre-absorber’. A pre-absorber can comprise dynamically adjustable material thicknesses, such as a plurality of plates of a given material (or variety of materials) than can be selectively placed in the beam's path. A pre-absorber typically consists of two wedges of attenuating materials (often graphite or beryllium) that slide against each other to vary the total tissue-equivalent pre-absorption beam-path. A conceptual pre-absorber design that could function for both deuteron and combined deuteron and proton operation is depicted diagrammatically in FIG. 7.
5. Modification of the range-modulator wheel. FIG. 8 shows a ‘pristine’ depth-dose curve for a mono-energetic proton beam. The dose from the patient's entrance point up to a depth just before the proton beam's maximum penetration depth is referred to as the ‘dose plateau’, which is relatively horizontal, and is much lower than the dose at the top of the ‘Bragg peak’ that occurs at the very end of the proton's range. If tumor dimensions were very small (for example, a few millimeters in size), using the energy-selector device described earlier combined with accurate imaging, the Bragg peak of the proton beam could be range-adjusted to fall exactly over the tumor volume. In the earliest days of proton therapy, the biological targets were typically small intracranial structures, and the proton Bragg peak was used in its pristine form to inactivate these structures³. However, since most tumor volumes are substantially larger in size than the width of a pristine Bragg peak, the approach of sweeping the Bragg peak back and forth to cover the depth dimension of the tumor volume was adopted. FIG. 9. shows how a series of pristine Brag peaks can be combined into a ‘spread-out Bragg peak’ (SOBP) to produce a relatively flat dose profile along the depth dimension of the tumor volume. To construct a SOBP requires the pristine Bragg peak of the proton beam to be moved to progressively different depths, while the overall depth of the SOBP requires a constant depth offset of all the constituent Bragg peaks. A range modulator wheel achieves both requirements. FIG. 10 (a) shows a diagram of a range modulator wheel. The small circle represents the footprint of the proton beam at a moment in time. As the range modulator wheel spins, the proton beam encounters steps of successively increasing thickness, which pre-absorb the proton beam by successively greater amounts, thus creating an array of depth-shifted Bragg peaks. To ensure that the summation of these Bragg peaks, i.e., the final SOBP, has a relatively flat dose profile, the intensity of each Bragg peak must be individually adjusted. This is done by varying the width of each of the range modulator wheel's steps: wider steps produce higher intensity Bragg peaks because the beam spends a longer time at that particular depth. FIG. 10 (b) shows a photograph of an actual range-modulator wheel. To obtain a wider SOBP (along the depth dimension), the range modulator wheel's axle is shifted perpendicularly to the axis of the proton beam so that the proton beam intercepts the wheel's steps at a smaller radius, where it encounters a larger change in adjacent step thickness, producing a wider SOBP. Similarly, the absolute thickness of the range modulator wheel's steps determines the depth at which the SOBP occurs, in essence functioning as a pseudo energy selector. For deuteron operation, a modified set of range-modulator wheels would need to be constructed.

A range modulation wheel (RMW) can be installed in the path of the ion beam within the irradiation field forming apparatus. The RMW has a plurality of blades extending radially from a rotary shaft, and distal ends of the blades are joined to a cylindrical member. The cylindrical member is concentric to the rotary shaft. Each blade has multiple steps, each having a different thickness and arranged successively in the circumferential direction of the RWM. Each step having a different thickness is extended from the rotary shaft to the cylindrical member. The ion beam propagating along the irradiation field forming apparatus passes those steps when passing through the rotating RMW. Therefore, the ion beam having passed the RMW has a plurality of energy components corresponding to the thickness of each of the steps through which the ion beam has passed. As a result, the dose of the ion beam is made uniform in the depth direction of the tumor in the patient body.

Alternatively, a combined charged particle beam scatterer/range modulator can comprise a fluid reservoir having opposing walls in a particle beam path, a drive to adjust the distance between the walls of the fluid reservoir, and a programmable controller for the drive to adjust the distance between the walls of the reservoir during exposure of a target to the beam. The distance between the opposing walls of the reservoir can be continuously adjustable. A first and second fluid reservoir can be arranged in series in the particle beam path. The first and the second reservoirs can independently contain high Z and low Z materials. The distance between the opposing walls of the first reservoir and, independently, the distance between the opposing walls of the second reservoir can be continuously adjustable.

An example of charged particle therapy system having a range modulation wheel for a charged particle beam, such as a proton or carbon ion beam, is disclosed in U.S. Pat. No. 7,355,189 (incorporated by reference).

6. Modification of the compensator. Compensators are designed and constructed individually for each patient such that SOBPs at different lateral and axial positions cover the tumor volume's lateral and axial shape profile. As mentioned above, it is the role of the range-modulator wheel to generate the SOBPs that are of the correct axial width. The role of the compensator is to place the SOBPs along corresponding beam trajectories at the correct depth, and also to shape the proton beam to the lateral profile of the tumor volume. Treatment planning staff, including physicians, physicists, and dosimetrists, determine the exact size, shape, and location of a patient's tumor volume based on CT and/or MRI imaging. An appropriate range-compensator is then constructed for each patient's individual treatment field.

Although the compensator designed for deuteron treatments would need to have a modified design compared to a proton compensator, since a set of compensators must be constructed for each individual patient's treatment, construction of a deuteron compensator in place of a proton compensator would not require additional effort nor a net increase in cost for the deuteron treatment.

The aperture is typically constructed of brass and controls the profile of the beam. It can be up to several inches thick and may measure from small to large in diameter for receipt into the snout of, for example, an IBA or a Still River proton therapy cyclotron. The aperture opening has a unique shape, masking the proton beam so that it is conformed to the desired lateral profile of the tumor.

Present proton beam therapy machines use two blocks, each which use a single-homogenous material, typically brass (aperture material) and typically acrylic (range compensator material), that has been shaped three-dimensionally and placed in sliding engagement with the slots of the snout. Careful registration or indexing of the radiation modulator (range compensator) and aperture material in the snout ensures that the proton beam's radiation dose is accurately delivered to the tumor volume.

7. Modification of treatment planning software. Since deuterons have never been employed for particle therapy, commercial treatment planning software is presently designed to handle only proton beams. To accommodate deuteron treatment planning, a number of software adjustments would need to be made, but such modifications would, in principle, be neither complicated nor costly.

### Radiobiological Implications of Using Deuterons.

In the early days of proton therapy, extensive in vitro and in vivo radiobiological studies were conducted on cell cultures, rodents, and dogs to determine the relationship between doses prescribed over many decades for megavoltage x-ray therapy and those that needed to be prescribed for proton therapy to achieve equivalent normal tissue responses. The question to be addressed is whether this extensive body of radiobiological research would need to be repeated if deuterons were to be used clinically. FIG. 10 shows a graph of ‘relative biological effectiveness’ (RBE) vs. ‘linear-energy-transfer’ (LET) for protons, deuterons, and alpha particles.⁴ RBE, when used as a multiplier of physical dose, results in a quantity called ‘RBE-weighted dose’. Expressing proton dose in units of RBE-weighted dose normalizes a tissue's biological response to protons to the same numerical values of x-ray dose. Therefore, once the RBE factor for proton beams was experimentally determined, radiation oncologists could conveniently continue to prescribe for proton treatments the same numerical doses they would have prescribed for megavoltage x-ray therapy, but in units of RBE-weighted dose rather than in units of dose per se. FIG. 10 shows RBE to be strongly dependent on a radiation's linear-energy-transfer (LET), which defines the spatial density of ionization produced along a charged particle's trajectory as it penetrates through tissue. In general, the higher the LET of the particle, the higher will be its RBE, although a maximum RBE is eventually reached after which further increase in LET results in decreasing RBE due to the so-called ‘LET overkill’ effect. This phenomenon is illustrated in FIG. 10, where the maximum attainable RBEs are shown for protons and deuterons.

In FIG. 10, two distinct RBE/LET domains can be identified: Domain 1 typically occurs in the vicinity of a particle's Bragg peak where RBE is at or near its maximum. In this domain, deuterons (open squares) and protons (solid circles) exhibit identical maximum RBE values of approximately 7.8. Domain 2 occurs in the plateau and spread-out-Bragg-peak (SOBP) regions of a particle's trajectory. In this domain, average LET values tend to be substantially lower. In domain 2, average LETs are typically less than 5 keV/μm, with correspondingly lower RBE values of approximately 1.0-1.2 for both protons and deuterons. Consequently, since in both domain 1 and domain 2 the RBEs of protons and deuterons are essentially identical, there should be no significant radiobiological differences between protons and deuterons. Experiments with inactivation of C3H10T1/2 cells by protons and deuterons found no difference in effectiveness of inactivation between protons and deuterons at two LET values in the range 10-20 keV/μ.⁵

Consequently, based both on theory and on direct cell inactivation experiments, the implementation of deuteron beams should not require any adjustment in clinical dose prescriptions compared to prescriptions historically developed for protons.

### Deuteron Break-Up and Pickup.

The proton and neutron that comprise a deuteron are held together by a relatively low nuclear binding energy of 2.2 MeV. Therefore, a deuteron accelerated to more than 2.2 MeV could, in principle, break up into its constituent proton and neutron. Alternatively, a deuteron may instead pick up a neutron or proton from the medium it traverses. Picking up a proton would create a particle consisting of two protons and a neutron, but this is a particle that cannot exist, so the only remaining option is for the deuteron to pick up a neutron. A deuteron combining with a neutron would create a ‘triton’, a radioactive beta-emitting nucleus of tritium with a half-life of 12.3 years. Since either of these two options would result in the disappearance of the deuteron—and, concomitantly, a deficit in the dose the deuteron beam would deliver at the target, it is important to determine how much potential dose perturbation each of these possible nuclear transformations could produce.

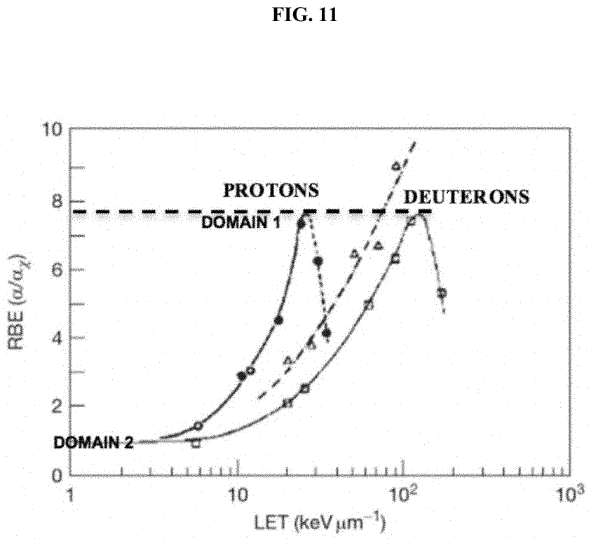

FIG. 11 shows the breakup, pickup, and total deuteron ‘disappearance’ (i.e., breakup+pickup) cross-sections for deuterons passing through a water-like transport medium.⁶ The abscissa is the deuteron energy in MeV-per-nucleon, and the ordinate is the microscopic cross-section in milli-barns (mB) for each of the two specific nuclear reactions referred to above. The solid vertical line intercepts the horizontal axis at 62.5 MeV/nucleon, which is the energy-per-nucleon of a 125 MeV deuteron (produced by a 250 MeV momentum-bound cyclotron). From FIG. 11, the deuteron breakup cross-section at 62.5 MeV/nucleon is 14 mB while the deuteron pickup cross-section at this same energy is 34 mB, as indicated by the horizontal solid lines.

The percent probability of a nuclear reaction occurring is given by the equation below as derived from Evans⁷:

Percent Probability=dₘₐₓ·100·ρ·Aν·σ/A

where σ is the nuclear reaction cross-section, Aν is Avogadro's Number, ρ is the density of the transport medium (tissue or water), A is the average atomic mass number of the transport medium (calculated as 13 for tissue or water), and dₘₐₓ is the penetration depth in tissue of a deuteron with an energy of 125 MeV.

Evaluating the above equation, the percentage probability of deuteron breakup is ˜0.4% and the percentage probability of deuteron pickup is ˜1%. Therefore, the consequences of deuteron breakup or deuteron pickup can be considered to be dosimetrically negligible.

Deuteron therapy proposes the use of a beam of deuterons instead of protons for certain disease conditions where substantially better dose conformity (i.e., a much lower amount of dose ‘spillover’ or beam ‘fuzziness’) would be desirable. The maximum depth of treatment for such a deuteron beam would be 66 mm, and its greatest utility would be to treat tumors that are relatively superficial and in contact with critical normal tissues that have elevated sensitivity to radiation dose. Under those constraints, it is exceptionally important to deliver therapy using particles that are more precise in geometric targeting than protons. One example of where a deuteron beam could be used to advantage would be the treatment of the ocular macula for wet macular age-related degeneration (AMD), a condition where overgrowth and leakage of macular blood vessels can result in progressive damage to the retina with subsequent catastrophic loss of vision. The target would be the macula, while the critical normal tissue to be avoided would be the retina surrounding the macula. In fact, a large number of other conditions exist that could be advantageously treated with deuterons rather than protons. The high dose conformity of a deuteron beam approaches that of a carbon ion beam, currently considered the ‘gold standard’ for dose conformity. However, unlike the exceedingly high cost of construction and operation of a carbon ion facility, deuteron therapy could either be implemented alongside proton therapy using relatively minor modifications to the same cyclotron and associated beam transport devices, or using a separate cyclotron. Concerns of deuteron breakup or pickup have been shown to be negligible with respect to dose perturbation.

Reference will now be made to the exemplified embodiments of the present general inventive concept, examples of which are illustrated in the accompanying drawings and illustrations. The exemplified embodiments are described herein in order to explain the present general inventive concept by referring to the figures.

The present disclosure provides a device, comprising an ion source and an energy selector device; wherein said ion source produces protons and deuterons.

In some embodiments, the ion source is a Penning ionization gauge (PIG).

In some embodiments, the ion source emits deuterons.

In some embodiments, the energy selector device is a pre-absorber.

In some embodiments, the energy selector device comprises two wedges of material configured to move with respect to each other in a way that allows for variable path distance of either a proton or a deuteron beam passing through the pre-absorber.

In some embodiments, the energy selector device comprises graphite.

In some embodiments, the energy selector device comprises beryllium.

In some embodiments, the present disclosure relates to any one of the aforementioned devices, further comprising a scattering foil.

In some embodiments, the scattering foil comprises lead.

In some embodiments, the scattering foil comprises nickel.

In some embodiments, the present disclosure relates to any one of the aforementioned devices, further comprising a range modulator wheel.

In some embodiments, the present disclosure relates to any one of the aforementioned devices, further comprising a compensator.

In some embodiments, the present disclosure relates to any one of the aforementioned devices, further comprising a cyclotron.

In some embodiments, the cyclotron is a momentum-bound cyclotron.

In v embodiments, the cyclotron is a synchrocyclotron.

In some embodiments, the PIG is positioned inside the cyclotron.

In some embodiments, the energy of the deuteron beam is from about 60 MeV to about 250 MeV. In some embodiments, the energy of the deuteron beam is from about 100 MeV to about 200 MeV. For example, the energy of the deuteron beam is about 125 MeV.

In some embodiments, the present disclosure relates to a method of treating a disorder, comprising administering to a tissue of a subject in need thereof an effective amount of a deuteron beam generated with the aid of any one of the aforementioned devices.

In some embodiments, the disorder is cancer.

In some embodiments, the tissue is cancerous.

In some embodiments, the cancer is selected from the group consisting of an ocular tumor, orbital tumor, lacrimal gland tumor, salivary gland tumor, intracranial falx meningioma, intracranial occipital meningioma, acoustic neuroma, shallow soft-tissue sarcoma, bone sarcoma of the extremities, neck lympho-nodal disease, chest wall desmoid tumor, abdominal wall desmoid tumor, breast cancer, pediatric rhabdomyosarcoma, and head and neck cancer.

In some embodiments, the disorder is head and neck cancer; and the head and neck cancer is selected from the group consisting of larynx cancer, thyroid cancer, salivary gland cancer, uveal melanoma, and retinal metastases.

In some embodiments, the deuteron beam is administered to an eye of the subject.

In some embodiments, the disorder is age-related macular degeneration, or a benign retinal tumor.

### REFERENCES CITED

1) Nuclear Energy Agency, 2008, http://www.oecd-nea.org/tools/abstract/detail/nea-0919/2)
2) Medical Physics, 2015, Vol. 42, No. 2, pp 1037-1047
3) Radiology, 1946, 47:487:1037-1047
4) Rad. Res., 1980, 82:277-289
5) International Journal of Radiation Biology, 1998, Volume 73, Issue 3
6) Transl. Cancer Res., 2017, Vol 6, Supplement 5
7) Robley Evans, The Atomic Nucleus, 1955, McGraw Hill Book Company, Inc., New York

### INCORPORATION BY REFERENCE

Each of the U.S. Patents and U.S. and International Patent Application Publications cited herein is hereby incorporated by reference in its entirety.

### EQUIVALENTS

Those skilled in the art will recognize, or be able to ascertain using no more than routine experimentation, many equivalents to the specific embodiments of the invention described herein. Such equivalents are encompassed by the following claims.

What is claimed is:

1. A device, comprising an ion source and an energy selector device, wherein said ion source produces protons and deuterons.

2. The device of claim 1, wherein the ion source is a Penning ionization gauge (PIG).

3. The device of claim 1, wherein the ion source emits deuterons.

4. The device of claim 1, wherein the energy selector device is a pre-absorber.

5. The device of claim 4, wherein the energy selector device comprises two wedges of material configured to move with respect to each other in a way that allows a variable path distance for either a proton or a deuteron beam passing through the pre-absorber.

6. The device of claim 1, wherein the energy selector device comprises graphite or beryllium.

7. The device of claim 1, further comprising a scattering foil.

8. The device of claim 7, wherein the scattering foil comprises lead or nickel.

9. The device of claim 1, further comprising a range modulator wheel.

10. The device of claim 1, further comprising a compensator.

11. The device of claim 1, further comprising a cyclotron.

12. The device of claim 11, wherein the cyclotron is a momentum-bound cyclotron.

13. The device of claim 1, further comprising a synchrocyclotron.

14. The device of claim 11, wherein the PIG is positioned inside the cyclotron.

15. The device of claim 1, wherein the energy of the deuteron beam is from about 60 MeV to about 250 MeV.

16. A method of treating a disorder, comprising administering to a tissue of a subject in need thereof an effective amount of a deuteron beam generated using a device of claim 1.

17. The method of claim 16, wherein the disorder is cancer.

18. The method of claim 16, wherein the cancer is selected from the group consisting of an ocular tumor, orbital tumor, lacrimal gland tumor, salivary gland tumor, intracranial falx meningioma, intracranial occipital meningioma, acoustic neuroma, shallow soft-tissue sarcoma, bone sarcoma of the extremities, neck lympho-nodal disease, chest wall desmoid tumor, abdominal wall desmoid tumor, breast cancer, pediatric rhabdomyosarcoma, and head and neck cancer.

19. The method of claim 18, wherein the disorder is head and neck cancer; and the head and neck cancer is selected from the group consisting of larynx cancer, thyroid cancer, salivary gland cancer, uveal melanoma, and retinal metastases.

20. The method of claim 16, wherein the disorder is age-related macular degeneration, or a benign retinal tumor.
